(19) United States
(12) Patent Application Publication
(10) Pub. No.: US 2004/0059270 A1
(43) Pub. Date: Mar. 25, 2004

(54) Pad and brace to counteract varus or valgus angulation

(76) Inventor: Gregg Berretta, Pointe Claire (CA)

Correspondence Address: OGILVY RENAULT, 1981 MCGILL COLLEGE AVENUE, SUITE 1600, MONTREAL, QC, H3A2Y3, CA

(21) Appl. No.: 10/664,018

(22) Filed: Sep. 17, 2003

(30) Foreign Application Priority Data
Sep. 19, 2002 (CA) 2,400,253

(51) Int. Cl. A61F 5/32; A61F 5/37

(52) U.S. Cl. 602/26; 602/23; 602/16

(57) ABSTRACT

A pad for attachment to a brace for relief of a varus or valgus osteo arthritic condition of the knee; the pad comprising a contoured shell, a shell lining, a single pivot point and pull strap fasteners; the pivot point located near the apex of the contoured lined shell; the pull strap fasteners located on both sides proximate the middle of the contoured shell.

### TECHNICAL FIELD

This invention is in the field of orthopedic knee braces. The invention relates to a brace for use by a person with an osteo arthritic condition which causes varus or valgus angulation of the leg.

### BACKGROUND OF THE INVENTION

In the prior art some pads are applied over the condular area which may cause skin irritation or skin breakdown. A pad applied against the condular area may result in rotation of the brace because of the small surface area of the pad in contact with the knee. Many prior art pads for osteo arthritic conditions required wrenches, screw drivers or air pumps to apply force to the medial or lateral portion of the knee. Other prior art means for relief of osteo arthritis by angulation are comprised of a single upright that can be adjusted by a screw mechanism to reduce the angulation of the leg.

### SUMMARY OF THE INVENTION

The invention relates to a pad and means of suspension of the pad on a brace to counteract the varus or valgus angulation of the leg. The top portion of the pad is pivotally connected at a single point to the medial or lateral femoral portion of the brace opposite the varus or valgus angulation. Posterior and anterior pull straps are fastened to the sides of the pad. The opposite free end of the posterior and anterior pull straps are passed through loops on the outside of the brace opposite the pad and tightened and fastened together to apply a counteracting force on the varus or valgus angulation. The pad of the instant invention may be tightened by the wearer by tightening the pull straps connected to the pad and fastening the pull straps by Velcro fasteners when the proper force has been applied to the pad. By using a large pad the force on the medial or lateral portion of the knee is spread over a larger area which will tend to prevent the brace from moving relative to the knee. The extension of the pad below the anterior and posterior straps is free so that the pad will provide an extended contact area with the medial or lateral portion of the knee. As the pad is only pivotally attached to the medial or lateral vertical femoral member of the brace at one point the pad will remain in contact with the medial or lateral portion of the femur and knee when the leg is moved.

### BRIEF DESCRIPTION OF THE DRAWINGS

FIG. 1 is a front view of a brace showing the top of the pad fastened by a pivot to a vertical portion of the femoral member of the brace.

FIG. 2 is an elevation view of the pad showing the top of the pad attached by a pivot to the interior of the vertical portion of the femoral member and each side of the pad fastened to the pull straps.

It will be noted that throughout the appended drawings, like features are identified by like reference numerals.

### DETAILED DESCRIPTION OF THE PREFERRED EMBODIMENT

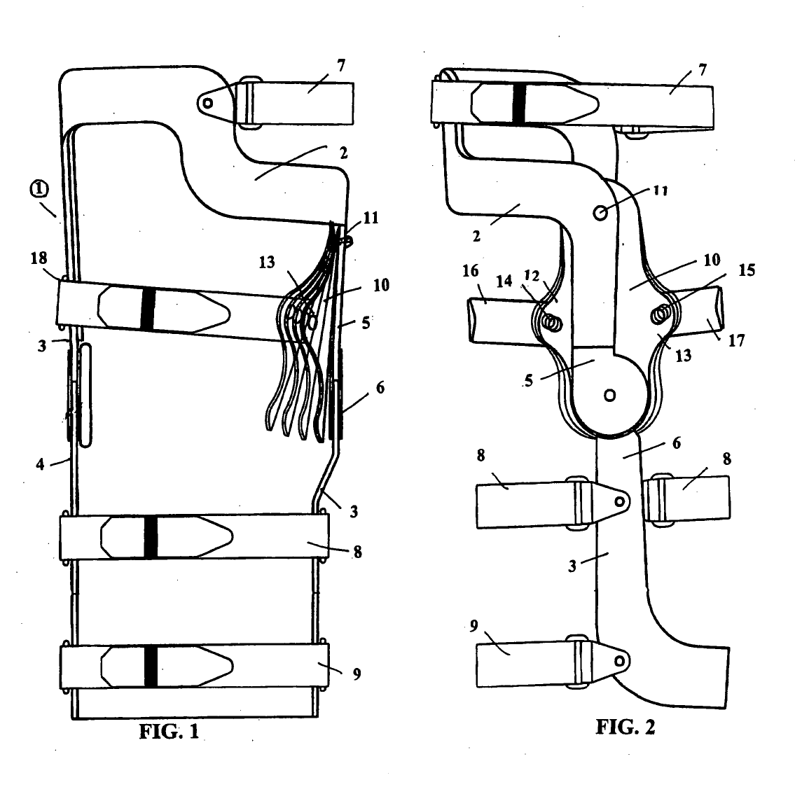

Referring to FIG. 1 there is shown a brace 1 having a femoral member 2, a tibial member 3. The femoral member 2 and tibial member 3 are pivotally connected together by lateral hinges 3, 4 and medial hinges 5, 6.

As seen in FIGS. 1 and 2 the femoral member 2 is fastened about the thigh by strap 7. The tibial member 3 is fastened about the calf by straps 8, 9 in a conventional manner.

The pad 10 is made of contoured plastic and is lined with soft foam. The pad 10 is fastened to the femoral member 2 by a pivot 11. The extended sides 12, 13 of the pad are fastened by fasteners 14 and 15 to pull straps 16, 17. One or both of the pull straps 16, 17 are slipped through a loop 18 to the side of the femoral member 3 on the side of the femoral member opposite to the pad 10.

During manufacture the pad 10 is fixed medially or laterally to the femoral member 2 by a pivotal fastener 11 depending on whether the osteo arthritic condition being corrected is varus or valgus angulation of the leg. As the pad 10 is connected to the femoral member at a single point by the pivotal fastener 11 the pull of the pad 10 gradually increases down the femur to the apex of the joint instability thereby extending pressure over an extended area of the medial or lateral portion of the knee. As the pad 10 is only pivotally connected to the femoral member 2 by pivotal fastener 11, the pad 10 acts independently within the rigid structure of the brace 1 reducing the tendency to cause rotation of the brace 1 on the leg. As the pad 10 is pivoted at a single point to the vertical femoral member the pad 10 is permitted to move both anteriorly and posteriorly when the user is walking maintaining a relatively constant force in the medial or lateral plane. The pull straps 16, 17 are fastened to the surface of one another enabling the user to adjust the amount of force to changing activity levels at any time without the use of a wrench, screw driver or air pump. The pull straps 16, 17 that extend from plastic contoured pad 10 are fully adjustable and may be trimmed to length. The pull straps 16, 17 when fastened also provide an added suspension feature by wrapping the supercondular area of the femur. The pad 10 applies pressure to the medial or lateral part of the knee over a well-distributed area. The pad 10 and pull straps 16, 17 can be used to correct varus or valgus osteo arthritic conditions.

I/we claim:

1. A pad for attachment to a brace for relief of a varus or valgus osteo arthritic condition of the knee;
the pad comprising a contoured shell, a shell lining, a single pivot point and pull strap fasteners;
the pivot point located near the apex of the contoured lined shell;
the pull strap fasteners located on both sides proximate the middle of the contoured shell.

2. A brace for relief of an osteo arthritic varus or valgus condition;
the brace comprising a femoral member, a tibial member, hinges, a contoured pad, pull straps, loops and pull strap fasteners;
the femoral member and tibial member connected by hinges;
the contoured pad comprised of a shell, a soft shell lining, a single pivot and strap fasteners;
the apex of the contoured pad is connected at a single point to the femoral member;
pull straps fastened to either side at the mid portion of the contoured tab;
loops to receive the pull straps located on the side of the femoral member opposite the pad,
fastening means on the pull straps to fasten the pull straps to one another when the pad is in the corrective position.
